(19) United States
(12) Patent Application Publication
(10) Pub. No.: US 2017/0360761 A1
(43) Pub. Date: Dec. 21, 2017

(54) Selective Inhibition of HDAC6 and HDAC8, and Methods of Use Thereof

(71) Applicant: Reaction Biology Corp., Malvern, PA (US)

(72) Inventors: Haiching Ma, Malvern, PA (US); Yuren Wang, Washington Crossing, PA (US)

(21) Appl. No.: 15/618,169

(22) Filed: Jun. 9, 2017

Related U.S. Application Data
(60) Provisional application No. 62/352,785, filed on Jun. 21, 2016.

(51) Int. Cl. A61K 31/428; A61K 45/06

(52) U.S. Cl. CPC A61K 31/428; A61K 45/06

(57) ABSTRACT

The present invention relates to methods of selectively modulating (for example inhibiting) the activity of histone deacetylase 6 and/or 8 (HDAC6 and/or HDAC8), and treating HDAC6 and/or HDAC8 associated diseases, including, for example, cancers, inflammatory disorders, and neurodegenerative disorders.

### CROSS-REFERENCE TO RELATED APPLICATIONS

The present application claims priority to U.S. Provisional Patent Application No. 62/352,785, filed Jun. 21, 2016, which is incorporated by reference herein in its entirety.

### BACKGROUND OF THE INVENTION

Histone deacetylase (HDAC) proteins are a family of enzymes that control the acetylation state of protein lysine residues, notably lysine residues contained in the N-terminal extensions of core histones. The acetylation state of histones affect gene expression by influencing chromatin conformation. In addition, the stability or biological function of several non-histone proteins is regulated by the acetylation state of specific lysine residues (Gallinari et al., 2007, Cell Res. 17:191-211; Kazantsev and Thompson, 2008, Nat Rev Drug Discov. 7:854-868).

In humans, HDAC proteins comprise a family of 18 members, which are separated into four classes based on size, cellular localization, number of catalytic active sites, and homology to yeast HDAC proteins. Class I includes HDAC1, HDAC2, HDAC3, and HDAC8. Class II consists of six HDAC proteins that are further divided into two subclasses. Class IIa includes HDAC4, HDAC5, HDAC7, and HDAC9, which each contain a single catalytic active site. Class IIb includes HDAC6 and HDAC10, which each contain two active sites, although only HDAC6 has two catalytically competent active sites. HDAC11 is the sole member of class IV, based on phylogenetic analysis. Class I, II, and IV HDAC proteins operate by a metal ion-dependent mechanism, as indicated by crystallographic analysis. In contrast, class III HDAC proteins, referred to as sirtuins (i.e., SIRT1 through SIRT7), operate by a NAD⁺-dependent mechanism unrelated to the other HDAC proteins (Gregoretti et al., 2004, J Mol Biol. 338:17-31; Grozinger and Schreiber, 2002, Chem Biol. 9:3-16).

The overexpression of different isoforms of HDACs has been found in several types of cancers, as well as in neurological and inflammatory pathologies. The use of HDAC inhibitors represents a treatment for such diseases (Valente and Mai, 2014, Expert Opin. Ther. Patents, 24:1-15; Falkenberg and Johnstone, 2014, Nat Rev Drug Discov. 13:673-91). The following are examples of HDAC inhibitors that have been tested in clinical trials both as single agents and in combination with chemotherapies and other targeted therapeutics: ACY1215 (Acetylon), CG200745 (Crystal Genomics), 4SC-202 (4SC corporation), CHR-2845 (Chroma Therapeutics), AR-42 (Amo Therapeutics), CUDC-101 (Curis Inc), Givinostat (Italfarmaco), Resminostat (4SC-Corporation), Pracinostat (S*BIO Pte Ltd), Etinostat (Syndax), Abexinostat (Pharmacyclics), Mocetinostat (Methylgene), Belinostat (TopoTarget), Valproic Acid (Instituto Nacional de Cancerologia), Panobinostat (Novartis), Vorinostat (Merck), and Romidepsin (Celgene).

HDAC inhibitors have been combined with a broad range of agents (Bots, & Johnstone, 2009. Clin. Cancer Res. 15, 3970-3977). The most prominent example of the empirical testing of HDAC inhibitors in combination is with DNA-damaging chemotherapeutics, which have led to many successful outcomes (Thum, et al, 2011, Future Oncol. 7, 263-283). HDAC inhibitors have also been successfully combined with DNMT inhibitors. Two Phase I trials have been carried out with vorinostat and bortezomib for the treatment of relapsing and/or refractory multiple myeloma with overall positive responses (Weber D M, Graef T et al 2012, Clin. Lymphoma Myeloma Leuk. 12, 319-324). A Phase III trial is currently assessing VPA (Valproic acid) in combination with levocamitine in children with spinal muscular atrophy (ClinicalTrials.gov identifier: NCT01671384). Vorinostat, panobinostat and VPA are currently being tested in combination with various antiretroviral therapies (ClinicalTrials.gov identifiers: NCT01680094, NCT01319383 and NCT01365065). A Phase I study combining Panobinostat with Ipilimumab to treat unresectable III/IV melanoma has just started (ClinicalTrials.gov identifiers: NCT02032810). HDAC6 specific inhibitors, rocilinostat (ACY-1215), is being tested clinically for the treatment of multiple myeloma in combination with bortezomib, following promising preclinical results (Santo L, Hideshima T, et al, 2012. Blood.; 119: 2579-2589.).

Many of the earlier HDAC inhibitors tested in clinical trials are either pan-inhibitors or have poor isoform selectivity. Thus, there is an interest in identifying HDAC inhibitors exhibiting selectivity within or between the human HDAC isoform classes. Achieving selectivity would not only reduce side effects, but would also provide the ability to target distinct therapeutic areas (Hu et al., 2003, J Pharmacol. Ther. 307: 720-728; Giannini et al., 2012, Future Med Chem. 4:1439-1460; Weïwer et al., 2013, Future Med Chem. 5:1491-1508; Falkenberg and Johnstone, 2014, Nat Rev Drug Discov. 13:673-91).

HDAC6 is a well-characterized class IIb deacetylase that regulates many important biological processes via the formation of complexes with its partner proteins. HDAC6 possesses two catalytic domains and a C-terminal zinc finger domain (ZnF-UBP domain, also known as BUZ) that binds free ubiquitin, as well as mono and polyubiquitinated proteins, with high affinity. HDAC6 is localized predominantly in the cytoplasm, and has been reported as a tubulin deacetylase that has effects on microtubule (MT)-mediated processes through both deacetylase-dependent and deacetylase-independent mechanisms. HDAC6 is important both for cytoplasmic and nuclear functions. Unlike other deacetylases, HDAC6 has unique substrate specificity for non-histone proteins such as α-tubulin, HSP90, cortactin, peroxiredoxins, chaperone proteins, β-Catenin, and hypoxia inducible factor-1α (HIF-1α) (Blackwell et al., 2008, Life Science 82:1050-1058; Shnakar and Sirvastava, 2008, Adv Exp Med Biol 615:261-298). HDAC6 also deacetylates protein peroxiredoxins, which are proteins critical in protecting cells from the oxidative effects of H₂O₂ (Parmigiani et al., 2008, PNAS 105:9633-9638). However, HDAC6 does not catalyze histone deacetylation in vivo. Therefore, it is a safer drug target since it does not impact DNA biology. As a MT-mediated cytoplasmic enzyme, HDAC6, through complexes with partner proteins, regulates multiple important biological processes, such as cell migration, cell spreading, immune synapse formation, viral infection, the degradation of misfolded proteins and stress granule (SG) formation. Mice lacking HDAC6 are viable and have greatly elevated tubulin acetylation in multiple organs. In addition, mice lacking HDAC6 exhibit a moderately impaired immune response and bone homeostasis. Such diverse functions of HDAC6 suggest that HDAC6 serves a potential therapeutic target for the treatment of a wide range of diseases. HDAC6 selective inhibitors have been tested in preclinical indications for cancers, neurology, inflammation, Gaucher's disease, Parkinson's disease, Huntington's disease; Alzheimer's diseases, depression and anxiety, and pain etc. (Gianniniet et al., 2012, Future Med Chem. 4:1439-1460; Falkenberg and Johnstone, 2014, Nat Rev Drug Discov. 13:673-91;).

HDAC8, on the basis of sequence homology, is considered to be a class I enzyme, although phylogenetic analysis has shown it to lay near the boundary of the class I and class II enzymes. HDAC8's importance has been revealed by knockdown experiments of selective HDAC isoforms showing it as essential for cell survival. HDAC8 specific inhibition selectively induces apoptosis in T-cell derived lymphoma and leukemic cells The expression of HDAC8 has been described in a variety of cancer entities e.g. colon, breast lung, pancreas and ovary cancer (Nakagawa et al. 2007, Oncol Rep, 18:769-774). In the highly malignant childhood cancer neuroblastoma high HDAC8 expression significantly correlates with poor prognostic markers and poor overall and event-free survival. In cultured neuroblastoma cells knockdown and pharmacological inhibition of HDAC8 resulted in inhibition of proliferation, reduced clonogenic growth, cell cycle arrest and differentiation (Oehme et al. 2009, Clin Cancer Res, 15:91-99). Furthermore, HDAC8 promotes lung, colon and cervical cancer cell proliferation and may regulate telomerase activity. The three dimensional crystal structure of human HDAC8 was the first to be solved, and 14 human HDAC8 structures co-crystallized with different inhibitors have been described. Currently, HDAC8 selective inhibitors are in preclinical trials for cancer (Giannini G et al., 2012, Future Med Chem. 4:1439-1460; Falkenberg and Johnstone, 2014, Nat Rev Drug Discov. 13:673-91). Thus, there remains a need in the art for inhibitors of HDACs having high selectivity within and between various HDAC classes, which can serve as therapeutic agents against a variety of diseases and disorders. The present invention fulfills this need.

Sirtuins 1-7 (SIRT1-7) belong to the third class of deacetylase enzymes, which are dependent on NAD(+) for activity. Sirtuins activity is linked to gene repression, metabolic control, apoptosis and cell survival, DNA repair, development, inflammation, neuroprotection, and healthy aging. Because sirtuins modulation could have beneficial effects on human diseases there is a growing interest in the discovery of small molecules modifying their activities. Sirtuin inhibitors with a wide range of core structures have been identified for SIRT1, SIRT2, SIRT3 and SIRT5 (splitomicin, sirtinol, AGK2, cambinol, suramin, tenovin, salermide, among others). SIRT1 inhibition has been proposed in the treatment of cancer, immunodeficiency virus infections, Fragile X mental retardation syndrome and for preventing or treating parasitic diseases, whereas SIRT2 inhibitors might be useful for the treatment of cancer and neurodegenerative diseases. (Villalba et al 2012, 38(5):349-59; Chen L, Curr Med Chem. 2011; 18(13): 1936-46).

Thus, there remains a need in the art for methods for selective inhibition of HDACs, in particular selective inhibition within and between various HDAC classes, methods which can serve as therapeutic basis against a variety of diseases and disorders. There remains a need in the art in particular for methods for selective inhibition of HDAC6 and/or HDAC8. The present invention fulfills this need.

### SUMMARY OF THE INVENTION

In one aspect, the invention relates to a method of selectively inhibiting a histone deacetylase enzyme (HDAC), comprising contacting the HDAC with a compound of Formula RBC-2008, or a salt or solvate thereof:

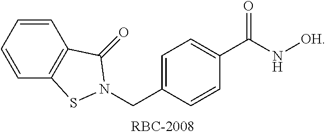 [SMILES: O=C(NO)c1ccc(Cn2sc3ccccc3c2=O)cc1]

In one embodiment, the HDAC is HDAC6. In another embodiment, the HDAC is HDAC8. In another embodiment, the compound is part of a composition at a concentration of less than 200 nM.

In another aspect, the invention relates to a method of treating a disease or disorder associated with an HDAC in a subject, comprising administering to the subject a therapeutically effective amount of a compound of Formula RBC-2008, or a salt or solvate thereof. In one embodiment, the HDAC is HDAC6. In another embodiment, the HDAC is HDAC8. In one embodiment, the subject is a human. In one embodiment, the disease or disorder is cancer. In one embodiment, the cancer is selected from the group consisting of multiple myeloma, leukemia, lymphoma, breast cancer, lung cancer, stomach cancer, liver cancer, blood cancer, bone cancer, pancreatic cancer, skin cancer, head or neck cancer, skin or eye melanoma, sarcoma of the uterus, ovarian cancer, rectal cancer, anal cancer, colorectal cancer, fallopian tube carcinoma, endometrium carcinoma, cervical cancer, small intestine cancer, endocrine gland cancer, thyroid cancer, parathyroid gland cancer, renal cell carcinoma, soft tissue sarcoma, urethra cancer, prostate cancer, bronchial cancer, myeloma, neuroma, and cutaneous squamous cell carcinoma. In another embodiment, the disease or disorder is a psychiatric disease or disorder. In another embodiment, the disease or disorder is a neurologic disease or disorder. In another embodiment, the disease or disorder is a neurodegenerative disease or disorder. In one embodiment, the neurodegenerative disease or disorder is selected from the group consisting of Alzheimer's disease (AD), Parkinson's disease (PD), and Huntington's disease (HD), frontotemporal dementia (FTLD), amyotrophic lateral sclerosis (ALS), and Charcot-Marie-Tooth disease (CMT). In another embodiment, the disease or disorder is a neuroinflammation disease or disorder. In one embodiment, the compound is administered to the subject orally, parenterally, intravascularly, intranasally, or intrabronchially. In one embodiment, the method further comprises administering to the subject a therapeutically effective amount of an additional therapeutic agent for the treatment of a disease or disorder. In some embodiments, the additional therapeutic agent is selected from the group consisting of an immunomodulatory drug, an immunotherapeutic drug, a DNA-damaging chemotherapeutic, a proteasome inhibitor, an anti-androgen receptor, an antiretroviral drug, a reverse-transcriptase inhibitor, a chemotherapeutic drug, and an immunosuppressant.

In another aspect, the invention relates to a method of immunomodulation for organ transplant, comprising administering to a patient a therapeutically effective amount of a compound of Formula RBC-2008, or a salt or solvate thereof.

In one aspect, the invention relates to a kit for selectively inhibiting an HDAC, comprising: an amount of a compound of Formula RBC-2008, or a salt or solvate thereof; and an instruction manual for the use thereof; wherein the HDAC is selected from the group consisting of HDAC6 and HDAC8.

In another aspect, the invention relates to a kit for treating a disease or disorder associated with an HDAC in a subject, comprising: an amount of a compound of Formula RBC-2008, or a salt or solvate thereof; and an instruction manual for the use thereof; wherein the HDAC is selected from the group consisting of HDAC6 and HDAC8.

In another aspect, the invention relates to a probe for imaging, diagnosing, or theragnosting a disease or disorder associated with an HDAC in a subject, comprising: a compound of Formula RBC-2008, or a salt or solvate thereof; wherein the HDAC is selected from the group consisting of HDAC6 and HDAC8.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts the compound of Formula RBC-2008 and various other compounds comparatively tested for HDAC inhibitory activity.

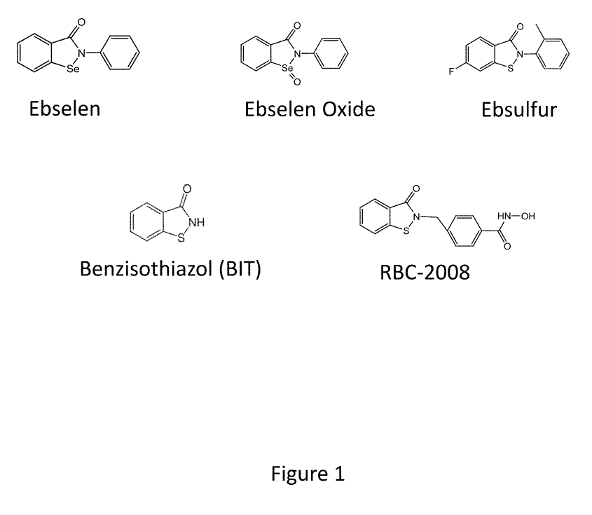

FIG. 2 is a table depicting comparative biochemical based assay IC₅₀ values of RBC-2008 and various other compounds against several HDAC human isoforms.

FIG. 3 is a chart depicting inhibition of HDACs by RBC-2008 in biochemical assays.

FIG. 4 is a chart depicting inhibition of Sirts by RBC-2008 in biochemical assays.

FIG. 5 is a chart depicting RBC-2008 activity on cell viability in human leukemia (CML) K-562 cell line.

FIG. 6 is a photograph depicting comparative modulation of α-tubulin acetylation in PC-3 cells.

FIG. 7 is a schematic depicting the HDAC Fluorescent Activity Assay, in which the deacetylation of a fluorogenic substrate by HDAC protein sensitizes it to the developer, which then generates a fluorophore having excitation at 360 nm light and emission at 460 nm, which is detected on a fluorometric plate reader.

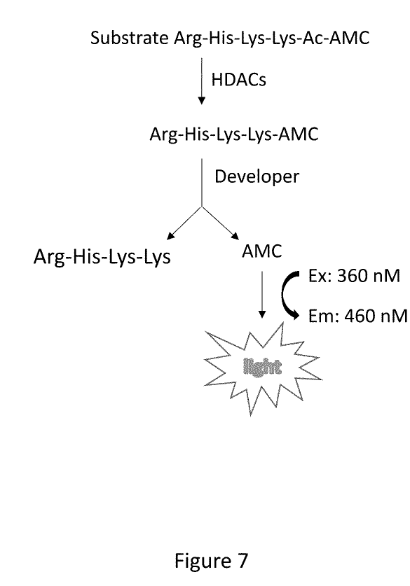

FIG. 8 is a graph of experimental data demonstrating the activity of RBC-2008 on the viability of human myeloma and leukemia cells.

FIG. 9, comprising FIGS. 9A-9B, depicts experimental data demonstrating the modulation of PD-L1 expression by RBC-2008 in U87MG cells. FIG. 9A is an image depicting the quantification of specific bands of interest. FIG. 9B is a graph depicting the quantification of specific bands of interest.

### DETAILED DESCRIPTION OF THE INVENTION

The present invention provides novel methods for selectively inhibiting HDACs, in particular HDAC6 and HDAC8, methods useful as therapies for various diseases and disorders, including but not limited to cancer, psychiatric disorders, neurologic disorders and neurodegenerative disorders, inflammation, virus infection, bone and muscle-related disorders such as cancer-induced cachexia.

### Definitions

As used herein, each of the following terms has the meaning associated with it in this section. Unless defined otherwise, all technical and scientific terms used herein generally have the same meaning as commonly understood by one of ordinary skill in the art to which this invention belongs. Generally, the nomenclature used herein and the laboratory procedures in biochemistry, analytical chemistry and organic chemistry are those well-known and commonly employed in the art. Standard techniques or modifications thereof are used for chemical syntheses and chemical analyses.

The articles “a” and “an” are used herein to refer to one or to more than one (i.e., to at least one) of the grammatical object of the article. By way of example, “an element” means one element or more than one element.

The term “about” as used herein when referring to a measurable value such as an amount, a temporal duration, and the like, is meant to encompass variations of ±20% or ±10%, more preferably ±5%, even more preferably ±1%, and still more preferably ±0.10%

The terms “patient,” “subject,” “individual,” and the like are used interchangeably herein, and refer to any animal, including mammals. In certain non-limiting embodiments, the patient, subject or individual is a human.

A “disease” is a state of health of an a subject wherein the subject cannot maintain homeostasis, and wherein if the disease is not ameliorated, the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health. As used herein, “treating a disease or disorder” means reducing the frequency and/or severity with which a symptom of the disease or disorder is experienced by an individual.

The term “treat,” as used herein, means reducing the frequency and/or severity of a sign or symptom of a disease or disorder experienced by a subject. Thus, “treat” and “treating” are not limited to the case where the subject (e.g., patient) is cured and the disease or disorder is eradicated. Rather, the present invention also contemplates treatment that merely reduces signs or symptoms, improves (to some degree) and/or delays disease or disorder progression. The term “treatment” also refers to the alleviation, amelioration, and/or stabilization of signs or symptoms, as well as a delay in the progression of signs or symptoms of a disease or disorder. As used herein, to “alleviate” a disease or disorder means to reduce the frequency and/or severity of one or more signs and/or symptoms of the disease or disorder.

The term “effective amount” in a subject, as used herein, refers to an amount that provides a therapeutic or prophylactic benefit in the subject. The term “therapeutically effective amount” refers to the amount of the compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the signs and/or symptoms of the disease or disorder being treated. The therapeutically effective amount will vary depending on the compound, the disease or disorder, the severity of the disease or disorder, and the age, weight, etc., of the subject to be treated.

The term “pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound or molecule useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “composition” refers to a mixture of at least one compound or molecule useful within the invention with one or more different compound, molecule, or material. As used herein “pharmaceutical composition” or “pharmaceutically acceptable composition” refers to specific examples of compositions, wherein at least one compound or molecule useful within the invention is mixed with one or more pharmaceutically acceptable carriers. In some instances, the pharmaceutical composition facilitates administration of the compound or molecule to a patient. Multiple techniques of administering a compound or molecule exist in the art including, but not limited to, intravenous, oral, aerosol, parenteral, ophthalmic, pulmonary and topical administration.

As used herein, an “instructional material” or “instruction manual” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range and, when appropriate, partial integers of the numerical values within ranges. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods of the Invention

In one aspect, the invention relates to a method of selectively inhibiting a histone deacetylase enzyme (HDAC), comprising contacting the HDAC with a compound of Formula RBC-2008, or a salt or solvate thereof:

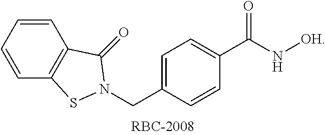 [SMILES: O=C(NO)c1ccc(Cn2sc3ccccc3c2=O)cc1]

In one embodiment, the HDAC is HDAC6. In another embodiment, the HDAC is HDAC8. In another embodiment, the compound is part of a composition at a concentration of less than 200 nM. In some embodiments, the compound of Formula RBC-2008 has a half maximal inhibitory concentration (IC₅₀) with respect to HDAC6 and/or HDAC8 of less than about 200 nM.

Another aspect of the present invention pertains to methods of treating a HDAC6 and/or HDAC8 associated disease or disorder in an individual, e.g., patient, by administering to the individual a therapeutically effective amount or dose of a compound of the present invention or a pharmaceutical composition thereof. In some embodiments, the individual has been diagnosed to have a HDAC6 and/or HDAC8 associated disease or disorder and is in need of treatment for the disease or disorder. An HDAC6 and/or HDAC8 associated disease can include any disease, disorder, or condition that is directly or indirectly linked to expression or activity of the HDAC6 and/or HDAC8, including over expression and/or abnormal activity levels. An HDAC6 and/or HDAC8 associated disease can also include any disease, disorder or condition that can be prevented, ameliorated, or cured by modulating HADC 6 and/or HDAC8 activity.

In some embodiments, the invention relates to methods of treating one or more diseases or disorders associated with the overexpression of one or more HDACs, in particular HDAC6 and HDAC8. In one embodiment, the invention provides a method of treating a disease or disorder related to the enzymatic control of the acetylation state of protein lysine residues, more specifically those contained in the N-terminal extensions of the core histones. In one embodiment, the invention provides a method of treating a disease or disorder associated with the overexpression of one or more HDACs, such as HDAC6 and/or HDAC8.

In one embodiment, the disease or disorder is cancer, such as, but not limited to, multiple myeloma, leukemia, lymphoma, breast cancer, lung cancer, stomach cancer, liver cancer, blood cancer, bone cancer, pancreatic cancer, skin cancer, head or neck cancer, skin or eye melanoma, sarcoma of the uterus, ovarian cancer, rectal cancer, anal cancer, colorectal cancer, fallopian tube carcinoma, endometrium carcinoma, cervical cancer, small intestine cancer, endocrine gland cancer, thyroid cancer, parathyroid gland cancer, renal cell carcinoma, soft tissue sarcoma, urethra cancer, prostate cancer, bronchial cancer, myeloma, neuroma, cutaneous squamous cell carcinoma, or the like.

In another aspect, the invention provides a method of treating a neurological disease or disorder. In another embodiment, the invention provides a method of treating an inflammatory disease or disorder. In other various embodiments, the diseases and disorders include, but are not limited to, diseases and disorders related to cell migration, cell spreading, immune synapse formation, viral infection, the degradation of misfolded proteins and stress granule (SG) formation. In other embodiments, the disease or disorder is an autoimmune diseases, such as rheumatoid arthritis, transplant rejection, and other inflammation indications. Other disease indications included are major genetic diseases such as sickle cell disease and thalassemia major (beta-thalassemia) and parasitic diseases such as malaria.

In yet another embodiment, the disease or disorder is a neurodegenerative disorder, such as Alzheimer's (AD), Parkinson's (PD) and Huntington's (HD), frontotemporal dementia (FTLD), amyotrophic lateral sclerosis (ALS) and Charcot-Marie-Tooth disease (CMT). In yet another embodiment, the disease or disorder is an autoimmune disease or disorder. In other various embodiments, the diseases and disorders treatable by the compound of the invention include, but are not limited to, diseases and disorders related to neurological disease, a neurodegenerative disorder, neuroinflammation, pain, epilepsy, Gaucher's disease, ischemic stroke, stroke, traumatic brain injury, allograft rejection, or a parasite related disease.

In another embodiment, the disease or disorder is a pathological autoimmune disorder such as juvenile oligoarthritis, collagen-induced arthritis, adjuvant-induced arthritis, Sjogren's syndrome, multiple sclerosis, experimental autoimmune encephalomyelitis, inflammatory bowel disease (for example, Crohn's disease, ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, autoimmune hemolytic anemia, pernicious anemia, and the like.

In another aspect, the invention provides a method of immunomodulation for organ transplant. In one embodiment, the method includes administering to a patient a therapeutically effective amount of compound RBC-2008.

In one embodiment, the method confers improved or superior retention of organ transplants.

### Prodrug Therapy

The invention includes methods comprising administration of prodrugs of RBC-2008. “Prodrug,” as used herein, means a compound which is convertible in vivo by metabolic means (e.g., by hydrolysis) to a compound of the present invention, i.e., RBC-2008. Various forms of prodrugs are known in the art, for example, as discussed in Bundgaard, (ed.), Design of Prodrugs, Elsevier (1985); Widder et al. (ed.), Methods in Enzymology, vol. 4, Academic Press (1985); Krogsgaard-Larsen et al. (ed). “Design and Application of Prodrugs,” Textbook of Drug Design and Development, Chapter 5, 113-191 (1991), Bundgaard et al., 1992, J. Drug Deliv. Rev. 8:1-38, Bundgaard, 1988, J. Pharm. Sci. 77:285 et seq.; and Higuchi and Stella (eds.), Prodrugs as Novel Drug Delivery Systems, American Chemical Society (1975). In one non-limiting example, the esters and amides of the alpha-carboxylic acid are prepared as prodrugs to improve oral bioavailability, whereby the ester or amide is stable in the stomach and gastrointestinal tract, is optimally transported across the lining of the gastrointestinal tract into the bloodstream, and is then converted by the ubiquitous esterases or amidases in the blood to the carboxylic acid moiety. In another non-limiting example, the ester prodrug is the methyl, ethyl, n-propyl or i-propyl ester. In another non-limiting example, the amide prodrug is the isopropyl amide or the 2,2,2-trifluoroethyl amide.

### Salts

The compound useful in the invention, i.e., RBC-2008, may form salts with acids or bases, and such salts are included in the present invention. In one embodiment, the salts are pharmaceutically-acceptable salts. The term “salts” embraces addition salts of free acids or free bases that are compounds useful within the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds useful in the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from the corresponding compound by reacting, for example, the appropriate acid or base with the compound.

### Combination Therapy

In one embodiment of a method of the invention, RBC-2008 is administered in combination with a second therapeutic agent for the treatment of a disease or disorder. In another embodiment, the second therapeutic agent is administered simultaneously, prior to, or after administration of the compound of the invention. In yet another embodiment, the second therapeutic agent is co-administered with RBC-2008. In yet another embodiment, the second therapeutic agent is co-administered and co-formulated with RBC-2008.

In some embodiments, one or more additional pharmaceutical agents can be used, such as, for example, immunomodulatory or immunotherapeutic drugs, such as immune checkpoint inhibitor monoclonal antibodies, thalidomide, lenalidomide (Len) and pomalidomide, steroids, such as dexamethasone, anticancer antibodies, such as nivolumab and ipilimumab, proteasome inhibitors, such as bortezomib, salinosporamide, anticancer drugs, such as romidepsin, and taxanes, oncolytic viral therapy agents, such as adenovirus, reovirus, or herpes simplex.

In one embodiment, the second therapeutic agent is a DNA-damaging chemotherapeutics such as idarubicin and cytarabine for the treatment of AML and MDS. In another embodiment, the second therapeutic agent is a proteasome inhibitor such as bortezomib for the treatment of relapsing and/or refractory multiple myeloma and lymphoma. In another embodiment, the second therapeutic agent is an anti-androgen receptor agent such as bicalutamide for the treatment of prostate cancer.

In some embodiments, the second therapeutic agent is an antiretroviral drug. In other embodiments, the second therapeutic agent is a reverse-transcriptase inhibitor. In other embodiments, the second therapeutic agent can be lamivudine, zidovudine, lopinavir, ritonavir, abacavir, tenofovir, emtricitabine, rilpivirine, efavirenz, elvitegravir, cobicistat, dolutegravir, darunavir, atazanavir, and raltegravir.

In certain embodiments, the compound of formula RBC-2008 may be administered to a subject in conjunction with (e.g., before, simultaneously, or following) any number of relevant treatment modalities including chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the compounds of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the compounds of the present invention are administered following B-cell ablative therapy such as agents that react with CD20, e.g., Rituxan.

### Dosing

The compound of formula RBC-2008, alone or in combination with another therapeutic agent, can be administered to a cell, a tissue, or a subject, to provide a therapeutic effect. Methods for the safe and effective administration of the compound of formula RBC-2008 are known to those skilled in the art. For instance, the administration of HDACs inhibitors is described in the literature.

Dosages of the compound of formula RBC-2008 range from about 0.1 μg/day to 10,000 mg/day, from about 1 μg/day to 1000 mg/day, and from about 10 μg/day to 100 mg/day, and any and all whole or partial increments there between.

Stated in terms of subject body weight, dosages range from about 0.1 μg/kg/day to about 1000 mg/kg/day, from about 10 μg/kg/day to about 500 mg/kg/day, from about 20 μg/kg/day to about 100 mg/kg/day, from about 50 μg/kg/day to about 50 mg/kg/day, and from about 0.10 mg/kg/day to about 5 mg/kg/day, and any and all whole or partial increments there between.

Oral dosages of the compounds of the invention range from about 0.1 μg/day to about 10,000 mg/day, from about 1 μg/day to about 1000 mg/day, from about 10 μg/day to about 100 mg/day, and from about 8 mg/day to about 80 mg/day, and any and all whole or partial increments there between.

Stated in terms of subject body weight, oral dosages range from about 0.1 μg/kg/day to about 1000 mg/kg/day, from about 10 μg/kg/day to about 500 mg/kg/day, from about 20 μg/kg/day to about 100 mg/kg/day, from about 50 μg/kg/day to about 50 mg/kg/day, and from about 0.10 mg/kg/day to about 5 mg/kg/day, and any and all whole or partial increments there between.

The compound of formula RBC-2008 can be administered in a dose range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, and any and all whole or partial increments there between.

In some embodiments, the dose of the compound of formula RBC-2008 is from about 0.0001 mg to about 25 mg. In some embodiments, a dose of the compound of formula RBC-2008 used in compositions described herein is less than about 100 mg, or less than about 80 mg, or less than about 60 mg, or less than about 50 mg, or less than about 30 mg, or less than about 20 mg, or less than about 10 mg, or less than about 5 mg, or less than about 2 mg, or less than about 0.5 mg. Similarly, in some embodiments, a dose of a second compound as described herein is less than about 1000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments there between.

### Pharmaceutical Composition

For administration of the compound of formula RBC-2008 to a subject, the compound can be suspended in any pharmaceutically acceptable carrier, for example, sterile water or buffered aqueous carriers, such as glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey), the disclosure of which is incorporated by reference as if set forth in its entirety herein.

The pharmaceutical compositions comprising the compound of formula RBC-2008 may be prepared, packaged, or sold in the form of a sterile injectable aqueous or oily suspension or solution. This suspension or solution may be formulated according to the known art, and may comprise, in addition to the active ingredient, additional ingredients such as dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

The compositions used in the methods of the invention are preferably administered to the subject as a pharmaceutical or veterinary composition, which includes systemic and topical formulations. Among these, preferred are formulations suitable for inhalation, or for respirable, buccal, oral, rectal, vaginal, nasal, intrapulmonary, ophthalmic, optical, intracavitary, intratraccheal, intraorgan, topical (including buccal, sublingual, dermal and intraocular), parenteral (including subcutaneous, intradermal, intramuscular, intravenous and intraarticular) and transdermal administration, among others. The route(s) of administration will be readily apparent to the skilled artisan and will depend upon any number of factors including the type and severity of the disease being treated, the type and age of the veterinary or human patient being treated.

The compositions used in the methods of the invention may be administered to the lungs of a subject by any suitable means, but are preferably administered by generating an aerosol or spray comprised of respirable, inhalable, nasal or intrapulmonarily delivered particles comprising the active compound, which particles the subject inhales, i.e., by inhalation administration. The respirable particles may be liquid or solid. Particles comprising the active compound for practicing the present invention should include particles of respirable or inhalable size; that is, particles of a size sufficiently small to pass through the mouth and larynx upon inhalation and into the bronchi and alveoli of the lungs. In general, particles ranging from about 0.05, about 0.1, about 0.5, about 1, about 1.5 to about 5, about 6, about 7, about 8, about 10 microns in size, more particularly particles about 0.5 to less than about 5 microns in size, are respirable or inhalable. When particles of nonrespirable size are included in the aerosol or spray, they tend to deposit in the throat and be swallowed. Thus, the quantity of non-respirable particles in the aerosol or spray is preferably minimized when intended for respirable administration or by inhalation. For nasal or intrapulmonary administration, a particle size in the range of about 10, about 11, about 15, about 20 to about 25, about 30, about 40, about 50, and sometimes even up to about 100 and about 500 microns is preferred to ensure retention in the nasal or pulmonary cavity. Pulmonary instillation is particularly useful in treating newborns.

Liquid pharmaceutical compositions used in the methods of the invention for producing an aerosol or spray may be prepared by combining the active compound with a stable vehicle, such as sterile pyrogen free water. Solid particulate compositions containing respirable dry particles of micronized active compound may be prepared by grinding dry active compound with a mortar and pestle, and then passing the micronized composition through a 400 mesh screen to break up or separate out large agglomerates. A solid particulate composition comprised of the active compound may optionally contain a dispersant which serves to facilitate the formation of an aerosol. A suitable dispersant is lactose, which may be blended with the active compound in any suitable ratio, e.g., a 1 to 1 ratio by weight. Other therapeutic and formulation compounds may also be included, such as a surfactant to improve the state of surfactant in the lung and to help with the absorption of the active agent.

Aerosols of liquid particles comprising an active compound may be produced by any suitable means, such as with a nebulizer. See, e.g., U.S. Pat. No. 4,501,729. Nebulizers are commercially available devices which transform solutions or suspensions of the active ingredient into a therapeutic aerosol mist either by means of acceleration of a compressed gas, typically air or oxygen, through a narrow venturi orifice or by means of ultrasonic agitation. Suitable compositions for use in nebulizer consist of the active ingredient in liquid carrier, the active ingredient comprising up to 40% w/w of the compositions, but preferably less than 20% w/w, and the carrier is typically water or a dilute aqueous alcoholic solution, preferably made isotonic with body fluids by the addition of, for example sodium chloride. Optional additives include preservatives if the composition is not prepared sterile, for example, methyl hydroxybenzoate, antioxidants, flavoring agents, volatile oils, buffering agents and surfactants.

Aerosols of solid particles comprising the active compound may likewise be produced with any sold particulate medicament aerosol generator. Aerosol generators for administering solid particulate medicaments to a subject produce particles which are respirable, as explained above, and they generate a volume of aerosol containing a predetermined metered dose of a medicament at a rate suitable for human administration. Examples of such aerosol generators include metered dose inhalers and insufflators.

Pharmaceutical compositions that are useful in the methods of the invention may be administered systemically in oral solid formulations, ophthalmic, suppository, aerosol, topical or other similar formulations. In addition to the compounds of the invention, or a biological equivalent thereof, such pharmaceutical compositions may contain pharmaceutically-acceptable carriers and other ingredients known to enhance and facilitate drug administration.

The pharmaceutical compositions described herein can be prepared alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

A pharmaceutical composition used in the methods of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition used in the methods of the invention will vary, depending upon the identity, size, and condition of the subject treated and further depending upon the route by which the composition is to be administered. By way of example, the composition may comprise between 0.1% and 100% (w/w) active ingredient. In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition used in the methods of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition used in the methods of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

In yet another embodiment, compositions used in the methods of the invention may be administered to the desired location of a subject by a transdermal patch. A transdermal patch is meant a system capable of delivery of a compound to a subject via the skin, or any suitable external surface, including mucosal membranes, such as those found inside the mouth. Such delivery systems generally comprise a flexible backing, an adhesive and a compound retaining matrix, the backing protecting the adhesive and matrix and the adhesive holding the whole on the skin of the subject. On contact with the skin, the compound-retaining matrix delivers the compound to the skin, the compound then passing through the skin into the subject's system.

Certain embodiments of the invention provide a pharmaceutical preparation/dosage formulation provided in the form of a transdermal patch and formulated for sustained release formulation, in a therapeutically effective amount sufficient to treat a disease associated with activation of an immune cell (e.g., rheumatoid arthritis) in a patient, wherein the dosage formulation, when administered (provided as a patch) to the patient, provides a substantially sustained dose over at least about 2 hours, 4 hours, 6 hours, 8, hours, 12 hours, 20 hours, or at least about 24 hours.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, bolus injections, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

A pharmaceutical composition used in the methods of the invention may be prepared, packaged, or sold in a formulation suitable for pulmonary administration via the buccal cavity. Such a formulation may comprise dry particles that comprise the active ingredient and that have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions used in the methods of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful in pulmonary delivery are also useful in intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition used in the methods of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to a subject, preferably a human, will vary depending upon any number of factors, including but not limited to, the type of animal and type of disease state being treated, the age of the subject and the route of administration.

The compound can be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the subject, and the like.

### Kit and Probes

In some embodiments, the present invention also includes pharmaceutical kits and/or research probes useful, for example, in the treatment or prevention of HDAC6 and/or HDAC8 associated diseases or disorders such as cancer, neurodegenerative diseases and pathological autoimmune response. In one embodiment, the kit includes a compound of the present invention. In one embodiment, the kit is useful for selectively inhibiting an HDAC. In one embodiment, the kit is useful for treating a disease or disorder associated with an HDAC. Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

In some embodiments, the present invention also includes probes comprising the compound of Formula RBC-2008, useful, for example, in the treatment or prevention of HDAC6 and/or HDAC8 associated diseases or disorders such as cancer, neurodegenerative diseases and pathological autoimmune response, or in the imaging or theragnostics approaches to HDAC6 and/or HDAC8 associated diseases or disorders such as cancer, neurodegenerative diseases and pathological autoimmune response. In one embodiment, a probe comprises the compound of Formula RBC-2008 further conjugated to a radiolabeled moiety, a fluorescent labeled moiety, or biotin. Any numbers of linkers known in the art can be used for conjugation. In another embodiment, no linker is necessary for conjugation. In some embodiments, a conjugated RBC-2008 probe is used for research, diagnostic and therapeutic purposes.

In one aspect, the invention provides methods comprising the use of theragnostics, or theranostics, further comprising the compound of Formula RBC-2008. Theragnostics, or theranostics, are compounds, formulations and compositions, capable of functioning as both therapeutic agents and diagnostic agents. For example, a probe of the invention can inhibit or modulate the activity of HDAC6 and/or HDAC8, and at the same time provide for the possibility of imaging HDAC6 and/or HDAC8 distribution in a cell, tissue, organ, or entire body. Modern approaches to theragnostics, or theranostics, have been described by Xie et al., 2010, Adv Drug Deliv Rev, 62(11):1064-1079, and Pene et al., 2009, Crit Care Med., 37(1 Suppl):S50-8, descriptions incorporated herein in their entirety.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Materials and Methods: Enzymes

Human HDAC1 (GenBank Accession No. NM_004964), full-length with a C-terminal His-tag and a C-terminal FLAG-tag, MW=56 kDa, was expressed in a baculovirus expression system.

Human HDAC2 (GenBank Accession No. NM_001527), full-length with a C-terminal His-tag, MW=56 kDa, was expressed in a baculovirus expression system.

Complex of human HDAC3 (GenBank Accession No. NM_003883), full-length with a C-terminal His tag, MW=49.7 kDa, and human NCOR2 (amino acid 395-489) (GenBank Accession No. NM_006312), N-terminal GST tag, MW=37.6 kDa, was co-expressed in a baculovirus expression system.

Human HDAC4 (GenBank Accession No. NM_006037), amino acids 627-1085 with an N-terminal GST tag, MW=75.2 kDa, was expressed in a baculovirus expression system.

Human HDAC5 (GenBank Accession No. NM_005474), full-length with an N-terminal GST tag, MW=150 kDa, was expressed in a baculovirus expression system.

Recombinant human HDAC6 (GenBank Accession No. BC069243), full-length, MW=180 kDa, was expressed by baculovirus in Sf9 insect cells using an N-terminal GST tag.

Human HDAC7 (GenBank Accession No. AY302468), (a.a. 518-end) with an N-terminal GST tag, MW=78 kDa, was expressed in a baculovirus expression system.

Human HDAC8 (GenBank Accession No. NM_018486), full-length with a C-terminal His tag, MW=46.4 kDa, was expressed in a baculovirus expression system.

Human HDAC9 (GenBank Accession No. NM_178423), amino acids 604-1066 with a C-terminal His tag, MW=50.7 kDa, was expressed in a baculovirus expression system.

Human HDAC10 (a.a. 1-481), GenBank Accession No. NM_032019 with an N-terminal GST tag and a C-terminal His tag, MW=78 kDa, was expressed in a baculovirus expression system.

Human HDAC11 (full length) (GenBank Accession No. NM_024827) with an N-terminal GST tag, MW=66 kDa, was expressed in a baculovirus expression system.

Human SIRT1 (Sirtuin 1, hSir2SIRT1) (GenBank Accession No. NM012238): Full length, MW=82 kDa, expressed in E. coli.

Human SIRT2 (Sirtuin 2, hSir2SIRT2) (GenBank Accession No. NM_012237): Full length, MW=43 kDa, expressed in E. coli.

Human SIRT3 (Sirtuin 3) (GenBank Accession No. NM_012239): Amino acids 102-399 (catalytically active), MW=32.7 kDa, expressed in E. coli.

Human SIRT5 (Sirtuin 5) (GenBank Accession No. NM_012241 (isoform 1); residues 37-310, MW=32.3 kDa) expressed in E. coli with an N-terminal His-tag).

The substrate RHKKAc-AMC, RHKAcKAc-AMC and AcK(trifluoroacetyl)-AMC were synthesized by Biomer.

ACY-1215, SAHA, Tubastatin A and Trichostatin A (TSA) were purchased from Selleckchem. TMP269 was purchased from MedKoo Biosciences. Nicotinamide adenine dinucleotide (NAD) was purchased from Tocris.

### Materials and Methods: Biochemical Assay Procedure

I. Compound handling: Testing compounds were dissolved in 100% DMSO to a specific concentration. The serial dilution was conducted by epMotion 5070 in DMSO.
II. HDAC reaction buffer: 50 mM Tris-HCl, pH8.0, 137 mM NaCl, 2.7 mM KCl, and 1 mM MgCl2, Added fresh: 1 mg/ml BSA, 1% DMSO.
III. Substrate: Fluorogenic HDAC General Substrate for HDAC1, 2, 3, 6, 10, 11 ans Sirt1, 2 and 3: Arg-His-Lys-Lys(Ac); HDAC8 only substrate: Arg-His-Lys(Ac)-Lys(Ac); Class2A Substrate (HDAC4, 5, 7 and 9): Acetyl-Lys(trifluoroacetyl)-AMC; Sirt5 substrate: Ac-Lys(succinyl)-AMC.
IV. General Reaction Procedure: (Standard IC50 determination)
   a. Delivered 2× enzyme in wells of reaction plate except No Enzyme (No En) control wells. Add buffer in No En wells.
   b. Delivered compounds in 100% DMSO into the enzyme mixture by Acoustic technology (Echo550; nanoliter range). Spin down and pre-incubation.
   c. Delivered 2× Substrate Mixture (Fluorogenic HDAC Substrate and co-factor (500 μM of Nicotinamide adenine dinucleotide (NAD⁺) in all Sirt assay) in all reaction wells to initiate the reaction. Spin and shake.
   d. Incubated for 1-2 hr. at 30° C. with seal.
   e. Added Developer with Trichostatin A (or TMP269 or NAD⁺) to stop the reaction and to generate fluorescent color.
   f. Fluorescence was read (excitatory, 360; emission, 460) using the EnVision Multilabel Plate Reader (Perkin Elmer)
   g. Endpoint reading was taken for analysis after the development reaches plateau.
V Data Analysis: The percentages of enzyme activity (relative to DMSO controls) and IC₅₀ values were calculated using the GraphPad Prism 4 program based on a sigmoidal dose-response equation.

### Example 1: Dose Dependent Inhibition of 15 HDACs by RBC-2008 in Enzymatic Assays, and IC₅₀ Values of RBC-2008 and Reference Compounds in the HDAC Enzymatic Assays

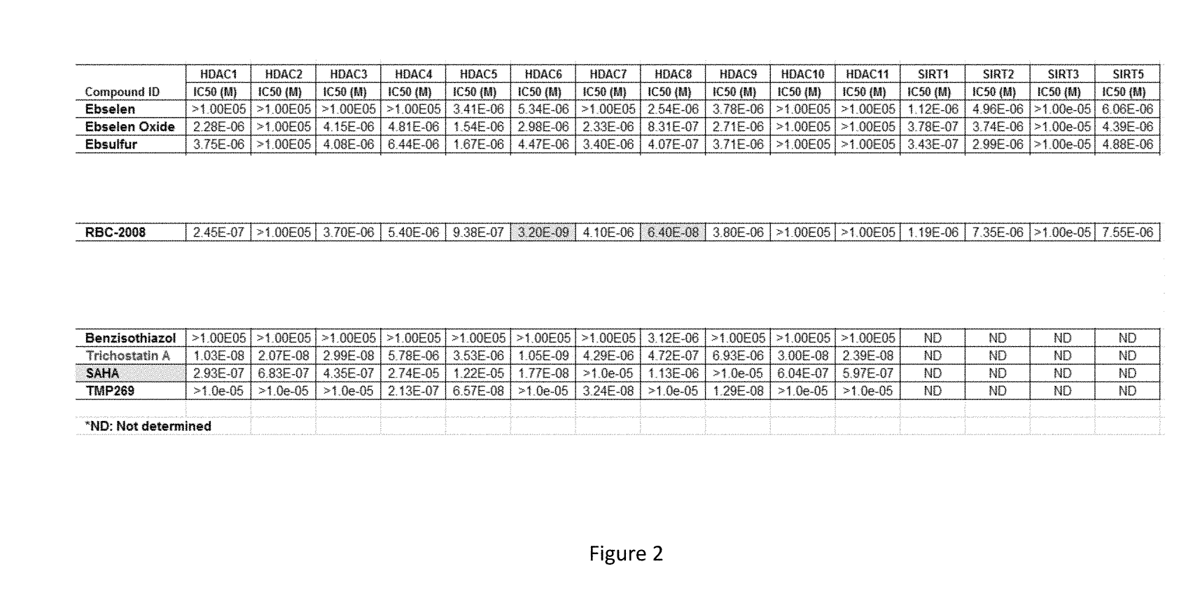

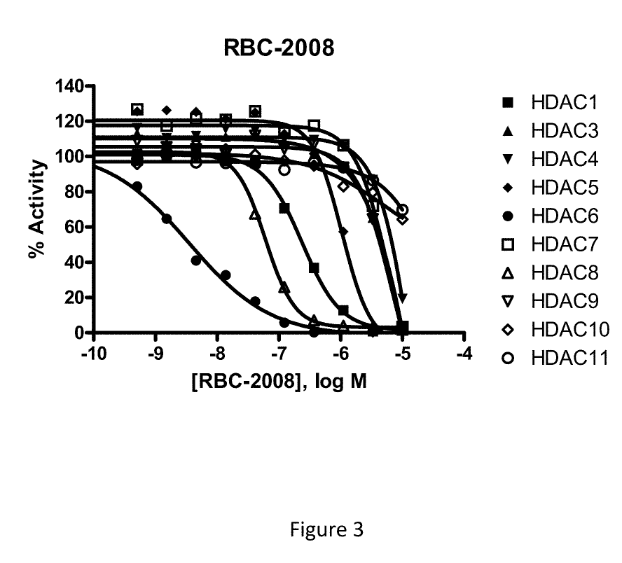

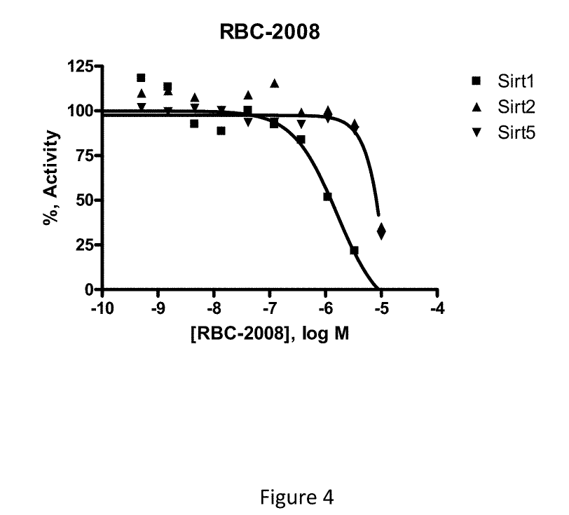

The inhibitory activities of RBC-2008 were determined using biochemical HDAC assays as depicted in FIGS. 2, 3 and 4. RBC-2008 with indicated doses was tested in the biochemical assays of HDAC1, HDAC2, HDAC3, HDAC4, HDAC5, HDAC6, HDAC7, HDAC8, HDAC9, HDAC10, HDAC11, SIRT1, SIRT2, SIRT3, and SIRT5 enzyme. The curve fit and IC₅₀ values were calculated using the GraphPad Prism 4 program based on a sigmoidal dose-response equation.

### Example 2: RBC-2008 Activity on Cell Viability in Human Leukemia (CML) K-562 Cell Line

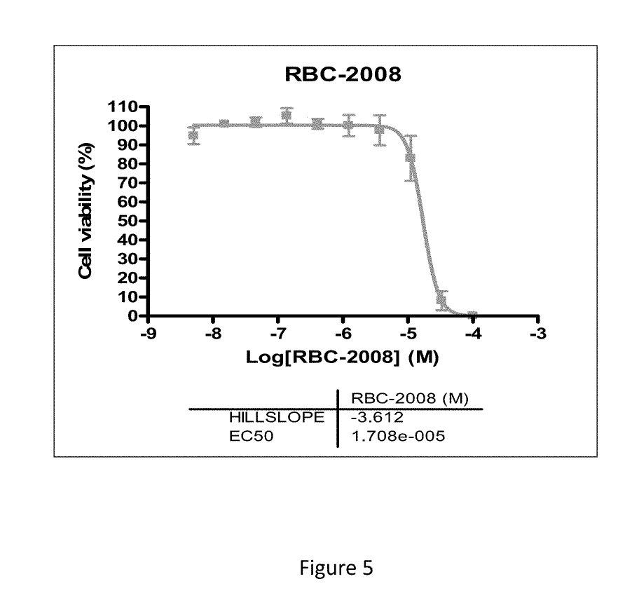

The effects of RBC-2008 on the viability of human tumor cell were assessed in an MTS assay. K562 cells were incubated with RBC-2008 in 384 well assay plates at 37° C., 5% CO₂ for 72 hours. 5 μl of CellTiter 96® AQueous One Solution Reagent (MTS assay reagent) from Promega was added to each well and incubated for 3 hours. Absorbance at 492 nm was recorded using an Envision Multilabel Reader. The IC₅₀ curves were plotted and the IC₅₀ values were calculated using the GraphPad Prism 4 program based on a sigmoidal dose-response equation (FIG. 5).

### Example 3: Modulation of a-Tubulin Acetylation by Compound RBC-2008 and Reference Compounds in PC-3 Cells

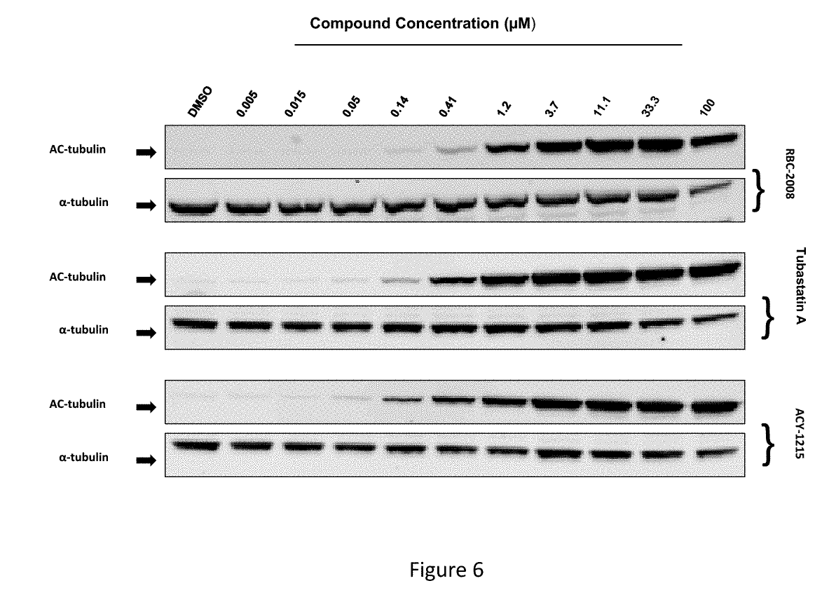

PC-3 cells were treated with the indicated concentrations of RBC-2008 for 24 hours as depicted in FIG. 6. The whole cell lysates were subjected to Western blot analyses with anti-Acetylated-tubulin antibody. The blots were then re-probed with anti-alpha-tubulin antibody.

### Example 4: Activity of Compound RBC-2008 on the Viability of Human Myeloma and Leukemia Cells

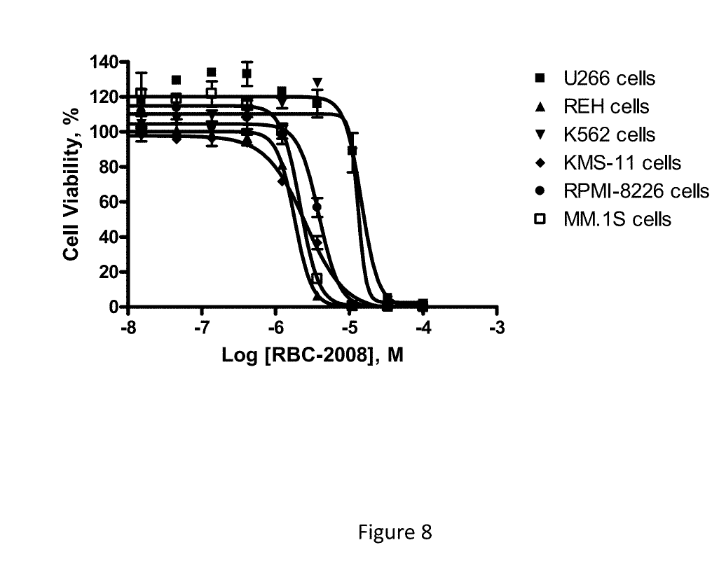

About 2000-5000 of U266 cells, REH cells, K562 cells, KMS-11 cells, RPMI-8226 cells and MM. 1S cells were incubated with RBC-2008 in 384 well assay plates at 37° C., 5% CO₂ for 72 hours. An amount of 5 μl of CellTiter 96® AQueous One Solution Reagent (MTS assay reagent) from Promega was added to each well and incubated for 3 hours. Absorbance at 492 nm was recorded by Envision Multilabel Reader. The IC₅₀ curves were plotted and the IC₅₀ values were calculated using the GraphPad Prism 4 program based on sigmoidal dose-response equation. The IC₅₀ values of RBC-2008 were calculated to be 14.5 μM, 1.8 μM, 13.1 μM, 2.5 μM, 3.8 M and 2.1 μM on U266 cells, REH cells, K562 cells, KMS-11 cells, RPMI-8226 cells and MM. 1S cells respectively (FIG. 8).

### Example 5: Modulation of PD-L1 Expression by Compound RBC-2008 in U87MG Cells

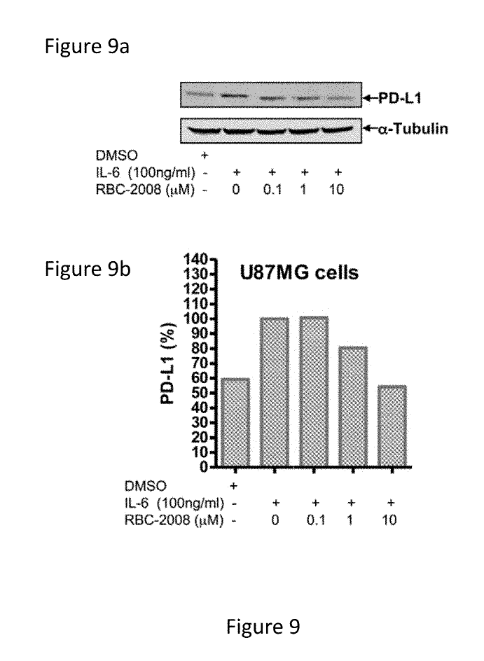

U87MG cells (American Type Culture Collection, Manassas, Va.) were grown in EMEM medium supplemented with 10% FBS. 100 μg/ml penicillin and 100 μg/ml streptomycin were added to the culture media. Cultures were maintained at 37° C. in a humidified atmosphere of 5% CO₂ and 95% air. The U87MG cells were starved in serum-free media for 24 h and then treated with compounds RBC-2008 at 0.1, 1, and 10 μM in the presence of 100 ng/ml of IL-6 for another 24 h. The media was removed and the cells were washed once with ice cold PBS. The cells were lysed with 1×RIPA buffer. The lysate samples were centrifuged at 12000 rpm for 10 minutes. The supernatants were transferred to a new set of eppendorf tubes. 4×LDS sample buffer plus 50 mM DTT was added to the cell lysates. 18 μl of cell lysate samples was subjected to SDS-PAGE with 12% Bis-Tris gel and transferred onto nitrocellulose membranes by iBlot dry blotting system. The membranes were blocked with LI-COR TBS blocking buffer for 1 h and then probed with anti-PD-L1 antibody and anti-α-Tubulin antibody overnight. Anti-rabbit IgG IRDye 680RD and anti-mouse IgG IRDye 800CW secondary antibodies were used to detect the primary antibodies. The membranes were scanned with LI-COR Odyssey Fc Imaging System. The specific bands of interest were quantified by LI-COR Image Studio Lite software (FIG. 9a), and a Bar graph was plotted using the GraphPad Prism 4 (FIG. 9b).

The disclosures of each and every patent, patent application, and publication cited herein are hereby incorporated herein by reference in their entirety. While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of selectively inhibiting a histone deacetylase enzyme (HDAC), comprising:
contacting the HDAC with a compound of Formula RBC-2008, or a salt or solvate thereof:

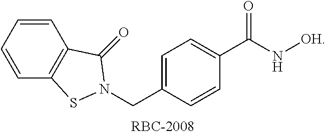 [SMILES: O=C(NO)c1ccc(Cn2sc3ccccc3c2=O)cc1]

2. The method of claim 1, wherein the HDAC is HDAC6.

3. The method of claim 1, wherein the HDAC is HDAC8.

4. The method of claim 1, wherein the compound, or the salt or solvate thereof, is part of a composition at a concentration of less than 200 nM.

5. A method of treating a disease or disorder associated with an HDAC in a subject, comprising:
administering to the subject a therapeutically effective amount of a compound of Formula RBC-2008, or a salt or solvate thereof:

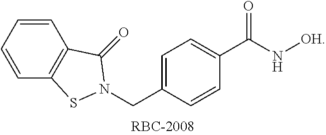 [SMILES: O=C(NO)c1ccc(Cn2sc3ccccc3c2=O)cc1]

6. The method of claim 5, wherein the HDAC is HDAC6.

7. The method of claim 5, wherein the HDAC is HDAC8.

8. The method of claim 5, wherein the subject is a human.

9. The method of claim 5, wherein the disease or disorder is cancer.

10. The method of claim 9, wherein the cancer is selected from the group consisting of multiple myeloma, leukemia, lymphoma, breast cancer, lung cancer, stomach cancer, liver cancer, blood cancer, bone cancer, pancreatic cancer, skin cancer, head or neck cancer, skin or eye melanoma, sarcoma of the uterus, ovarian cancer, rectal cancer, anal cancer, colorectal cancer, fallopian tube carcinoma, endometrium carcinoma, cervical cancer, small intestine cancer, endocrine gland cancer, thyroid cancer, parathyroid gland cancer, renal cell carcinoma, soft tissue sarcoma, urethra cancer, prostate cancer, bronchial cancer, myeloma, neuroma, and cutaneous squamous cell carcinoma.

11. The method of claim 5, wherein the disease or disorder is a psychiatric disease or disorder.

12. The method of claim 5, wherein the disease or disorder is a neurologic disease or disorder.

13. The method of claim 5, wherein the disease or disorder is a neurodegenerative disease or disorder.

14. The method of claim 13, wherein the neurodegenerative disease or disorder is selected from the group consisting of Alzheimer's disease (AD), Parkinson's disease (PD), and Huntington's disease (HD), frontotemporal dementia (FTLD), amyotrophic lateral sclerosis (ALS), and Charcot-Marie-Tooth disease (CMT).

15. The method of claim 5, wherein the disease or disorder is a neuroinflammation disease or disorder.

16. The method of claim 5, wherein the compound is administered to the subject orally, parenterally, intravascularly, intranasally, or intrabronchially.

17. The method of claim 5, further comprising:
administering to the subject a therapeutically effective amount of an additional therapeutic agent for the treatment of a disease or disorder.

18. The method of claim 17, wherein the additional therapeutic agent is selected from the group consisting of an immunomodulatory drug, an immunotherapeutic drug, a DNA-damaging chemotherapeutic, a proteasome inhibitor, an anti-androgen receptor, an antiretroviral drug, a reverse-transcriptase inhibitor, a chemotherapeutic drug, and an immunosuppressant.

19. A method of immunomodulation for organ transplant, comprising:
administering to a patient a therapeutically effective amount of a compound of Formula RBC-2008, or a salt or solvate thereof: 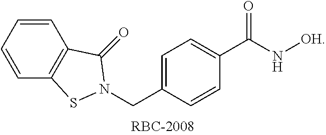 [SMILES: O=C(NO)c1ccc(Cn2sc3ccccc3c2=O)cc1]
